(19) United States
(12) Patent Application Publication
(10) Pub. No.: US 2026/0183357 A1
(43) Pub. Date: Jul. 2, 2026

(54) Amino Acid and Herbal-Based Compositions

(71) Applicant: Pure Land Wellness LLC, Sacramento, CA (US)

(72) Inventors: Li-Sann Mullings, Thousand Oaks, CA (US); Amanda Vickers Souther, Candler, NC (US)

(21) Appl. No.: 19/437,282

(22) Filed: Dec. 30, 2025

Related U.S. Application Data
(60) Provisional application No. 63/739,930, filed on Dec. 30, 2024.

(51) Int. Cl. A61K 36/258; A61K 31/198; A61K 31/455; A61K 33/30; A61K 36/31; A61P 15/00

(52) U.S. Cl. CPC A61K 36/258; A61K 31/198; A61K 31/455; A61K 33/30; A61K 36/31; A61P 15/00

(57) ABSTRACT

A composition comprising an amino acid component selected from L-Arginine, Citrulline Malate, L-Theanine, or any combination thereof, and an herbal and/or vitamin component selected from at least two of Niacin, Panax Ginseng, Lepidium Meyenii, Turnera Diffusa, Ptychopetalum Olacoides, Zinc, or any combination thereof.

### CROSS-REFERENCE TO RELATED APPLICATION

This application claims the benefit of the priority date of U.S. Provisional Application Ser. No. 63/739,930, filed on Dec. 30, 2024, the contents of that application being incorporated by reference herein in their entirety and for all purposes.

### FIELD OF THE INVENTION

This invention relates to compositions comprising synergistic combinations of amino acids and herbal ingredients and more particularly to the use of these compositions for the treatment of sexual performance disorders.

### BACKGROUND OF THE INVENTION

There is desire for medical solutions that treat health disorders in a more holistic manner, in other words improving a patient's overall mental, physical, and emotional vitality in an effort to reduce health issues. This is particularly true with disorders related to sexual performance and pleasure enhancement. Most conventional treatments are “in-the-moment” solutions as opposed to more holistic solutions that address underlsecondg causes of sexual performance issues.

### SUMMARY OF THE INVENTION

The present invention contemplates a composition comprising an amino acid component selected from L-Arginine, Citrulline Malate, L-Theanine, or any combination thereof, and an herbal and/or vitamin component selected from at least two of Niacin, Panax Ginseng, Lepidium Meyenii, Turnera Diffusa, Ptychopetalum Olacoides, Zinc or any combination thereof.

The composition may be effective for treatment of sexual performance health conditions.

### DETAILED DESCRIPTION

The explanations and illustrations presented herein are intended to acquaint others skilled in the art with the present teachings, its principles, and its practical application. The specific embodiments of the present teachings as set forth are not intended as being exhaustive or limiting of the present teachings. The scope of the present teachings should be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description. Percentages herein refer to weight percent, unless otherwise indicated.

In general, the teachings herein contemplate a composition formulated to improving mental, physical, and emotional vitality, sexual performance, and pleasure enhancement. The compositions described herein are synergistic blends in the form of dietary supplements, functional food systems, or a combination thereof that address the root causes of male sexual performance issues, and decreased libido in both males and females.

The compositions described herein comprise amino acids, herbal components, bioavailable mineral compounds, and combinations thereof (e.g., ingredients or components).

The compositions may comprise a combination of adaptogens, vasodilators, mineral cofactors, and combinations thereof.

The compositions described are formulated to improve one or more of physical stamina, mental focus, emotional confidence, and circulatory health.

The compositions described are formulated to promote one or more of relaxation, stress resilience, and long-term hormonal health for users.

By addressing underlsecondg issues such as poor circulation, hormonal imbalances, low energy, and stress-related performance challenges, the compositions described herein provide a comprehensive solution. This root-cause-focused methodology is aimed at unlocking lasting transformation rather than offering temporary fixes.

The components described below may be included in the compositions described herein. It is possible that a particular composition include only one of the following components. It is possible that a particular composition include all of the following components. It is possible that the compositions include a blend of one or more of the components listed below. It is also possible that each component is provided in a separate capsule, powder, or other format, requiring a user to ingest each component individually.

Velvet is also known as deer antler. The velvet may be ingested in an amount of 100 to 1000 mg, or even 300-500 mg. Velvet may utilized as a foundational component of the compositions described herein. Velvet may improve vitality, boost testosterone, and/or enhance physical stamina.

Turnera diffusa is also known as Damiana. The Turnera diffusa may be ingested in an amount of 100 to 1000 mg, or even 200-400 mg. Turnera diffusa may enhance libido and sexual pleasure by modulating dopamine levels and reducing anxiety.

Epimedium is also known as horny goat weed. Epimedium may be ingested in an amount of 100 to 1000 mg, or even 250-500 mg. The active ingredient in epimedium is icariin which may increase nitric oxide levels, leading to improved blood flow and erectile function. It is possible that epimedium may be ingested after velvet and/or in specific ratios with velvet to improve overall function of the velvet, the epimedium, or both.

Withania somnifera is also known as ashwagandha. Withania somnifera may be ingested in an amount of 100 to 1000 mg, or even 300-500 mg. Withania somnifera may reduce cortisol levels and improve endurance and vitality. Withania somnifera may be paired with selected adaptogens an/or stamina-enhancing herbal supplements.

Lepidium meyenii is also known as Maca. Lepidium meyenii may be ingested in an amount of 100 to 2000 mg, or even 500-1000 mg. Lepidium meyenii may increase energy levels, fertility, and/or libido. Lepidium meyenii may be incorporated into the composition an/or ingested after other components to provides for sustained energy levels.

Ginkgo biloba may be included in the compositions described herein. Ginkgo biloba may be ingested in an amount of 100 to 1000 mg, or even 120-240 mg. Ginkgo biloba may enhance peripheral circulation and cognitive function, improving erectile function by increasing blood flow to the genital region.

Tribulus terrestris may be included in the compositions described herein. Tribulus terrestris may be ingested in an amount of 100 to 2000 mg, or even 300-600 mg. Tribulus terrestris may boost testosterone and libido. Tribulus may be incorporated into the composition an/or ingested after other components to support hormone production and balance.

Panax ginseng may be included in the compositions described herein. Panax ginseng may be ingested in an amount of 100 to 1000 mg, or even 200-400 mg. Panax ginseng is an adaptogen and may improve physical stamina. It is possible that panax ginseng may be incorporated in the compositions in specific ratios with Tribulus terrestris to improve overall function of the panax ginseng, the Tribulus terrestris, or both.

Mondia whitei may be included in the compositions described herein. Mondia whitei may be ingested in an amount of 100 to 2000 mg, or even 300-600 mg. Mondia whitei is a strong adaptogen and may be utilized for circulatory benefits, and for reinforcing overall stamina.

L-Arginine may be included in the compositions described herein. L-Arginine is a precursor to nitric oxide (NO) and may support vasodilation, which is critical for erectile function and enhanced blood flow. L-Arginine may be ingested in an amount of 100 to 3000 mg, or even 1000-2,000 mg per day. L-Arginine may serve as the foundation for one or more compositions described herein. It may be preferred to blend L-Arginine first since it is a stable amino acid that combines well with citrulline malate in order to potentiate citrulline malate's effects on nitric oxide production.

Citrulline malate may be included in the compositions described herein. Citrulline malate may be converted into L-Arginine in the body and may help sustain NO levels longer than L-Arginine alone, improving endurance and blood flow. Citrulline malate may be ingested in an amount of 100 to 3000 mg, or even 1500-2,000 mg per day. Citrulline malate may be blended with L-Arginine early in the formulation, as it may complement L-Arginine in promoting blood flow and vasodilation effects. Combining these two helps achieve a consistent and sustained NO boost for vascular health and erectile support.

Niacin may be included in the compositions described herein. Niacin may aid blood flow by dilating blood vessels, enhancing the NO effects of L-Arginine and citrulline alate. It may also contribute to cholesterol management and energy metabolism. Niacin may be ingested in an amount of 1 to 200 mg, or even 15-50 mg per day. Niacin may be incorporated into the compositions described herein after the NO-boosting amino acids. Niacin may further improve blood vessel dilation and circulation. Care should be taken with Niacin, as doses too high can cause flushing; a dose around 25 mg can achieve effective results with minimal side effects.

Panax ginseng may be added to the compositions described herein after the NO-boosting ingredients as it may work on a different pathway to stimulate libido and stamina. Panax ginseng may also blend well with other adaptogens, like Lepidium meyenii, for additional vitality and mood support. Lepidium meyenii may be added to the compositions described herein after the panax ginseng for a complementary adaptogenic effect. Lepidium meyenii may have little to no effect on NO pathways, making it a useful addition without deleterious effects on the overall function of the compositions described herein.

Ptychopetalum olacoides may be included in the compositions described herein. Ptychopetalum olacoides may enhance libido, reduce fatigue, and supporting nervous system health. Ptychopetalum olacoides may be ingested in an amount of 50 to 1000 mg, or even 200-400 mg per day. Ptychopetalum olacoides and Turnera diffusa together may form a synergistic blend to support both physical and mental aspects of sexual health.

Zinc may be included in the compositions described herein. Zinc plays a critical role in testosterone synthesis, sperm production, and overall male reproductive health. Zinc may be ingested in an amount of 1 to 30 mg, or even 10-15 mg per day. As an essential mineral, Zinc is best added toward the end of the formulation process. It is important to keep within recommended daily amounts, as excessive zinc can interfere with copper absorption and cause stomach upset.

L-Theanine may be included in the compositions described herein. L-Theanine may promote relaxation without drowsiness, helping to counteract any potential overstimulation from the formulation. L-Theanine may also improve mood by enhancing dopamine and serotonin levels. L-Theanine may be ingested in an amount of 30 to 800 mg, or even 100-200 mg per day. L-Theanine may be added to the compositions described herein last, as it may provide a calming effect that can help balance out the stimulating properties of the formula, creating a smoother, balanced effect on mood and mental clarity.

### Processing

As one non-limiting example, L-Arginine and citrulline malate may be combined first, in the primary stage of formulation. These amino acids may form the base of the formula for NO production and improved blood flow. Niacin may then be added to synergistically enhance blood flow and vasodilation. It is possible that the niacin may cause flushing, so blending should occur carefully and in an amount of less than 40 mg or even less than 30 mg but greater than 10 mg, or greater than 20 mg (e.g., around 25 mg). Panax ginseng and Lepidium meyenii may then be added to promote energy and libido through adaptogenic and hormonal support.

Turnera diffusa and Ptychopetalum olacoides may then be added for their complementary effects on libido and mood. Turnera diffusa may help with relaxation, while Ptychopetalum olacoides supports both physical and mental sexual health. Zinc may then be added to enhance male reproductive health and testosterone production. Lastly, L-Theanine can be added to smooth out the formula's effects on energy and mood, providing a relaxing balance to the stimulatory components.

### Example 1—the Following Composition is an Exemplary Composition

| Ingredient | Amount | |
|---|---|---|
| L-Arginine | 1,000-2,000 | mg |
| Citrulline Malate | 1,500-2,000 | mg |
| Niacin | 15-25 | mg |
| Panax Ginseng (standardized to 5% | 100-200 | mg |
| ginsenosides) | | |
| Lepidium Meyenii | 500-1,500 | mg |
| Turnera Diffusa | 200-500 | mg |
| Ptychopetalum Olacoides | 200-400 | mg |
| Zinc | 10-15 | mg |
| L-Theanine | 100-200 | mg |

First ingredients are those that stimulate, energize, and catalyze. These typically boost testosterone, circulation, and physical vitality. Second ingredients are those that that harmonize, nourish, and sustain. These often stabilize hormones, calm the nervous system, and replenish vital energy reserves. By pairing first ingredients with complementary second counterparts, the resulting formulation can address both the root cause and symptomatic manifestations of a condition. This creates a more integrative approach to healing.

It may be possible to combine first ingredients for immediate energy, libido, or arousal effects. These first ingredients can be paired them with second ingredients for stress reduction, hormone stabilization, or long-term nourishment.

First and second ingredients may be utilized in ratios of about 60% by weight first ingredients to about 40% by weight second for a more energizing formula. For a calming yet potent blend, reverse the ratio to about 40% by weight first ingredients and 60% by weight second ingredients.

Acute Energy and Arousal: Prioritize first ingredients like Yohimbe and L-Arginine paired with lighter second adaptogens like Maca. Chronic Support and Rejuvenation: Use deep-nourishing second ingredients like Rehmannia or Shilajit paired with slow-acting first ingredients like Tribulus or Red Ginseng.

By pairing second and first ingredient pairs, it is possible to ensure that the body doesn't overstimulate (first ingredient excess) or underperform (second ingredient deficiency).

### Example Pairings

L-Arginine Pairings: L-Theanine: Counters the excitatory effects of L-Arginine, helping to prevent overstimulation and performance anxiety. Shilajit: Deeply replenishes minerals, supporting the body during nitric oxide-driven vasodilation.

Citrulline Malate Pairings: Rehmannia Root: Protects against kidney strain from increased circulation and complements Citrulline's vascular benefits. Ashwagandha: Enhances physical and mental stamina, offering stress modulation alongside Citrulline's endurance boost.

Niacin Pairings: Hawthorn Berry: Adds a calming effect on the heart and vessels, preventing flushing side effects. Cordyceps Mushroom: Amplifies energy and oxygen uptake while supporting Niacin's vascular health benefits.

White Panax Ginseng Pairings: Schisandra Berry: Provides adaptogenic Second support to balance Ginseng's energizing effects, enhancing focus and sexual vitality. Gotu Kola: Supports circulation and cognitive clarity, reducing stress and anxiety.

Maca Root Pairings: Tribulus terrestris: Complements Maca's energy and libido benefits by adding testosterone-boosting properties. Epimedium: Combines with Maca's hormonal stability to create a highly potent libido-enhancing blend.

Damiana Pairings: Muira Puama: Adds First stimulation to Damiana's mood-enhancing Second, creating a balanced aphrodisiac. Yohimbe Bark: Enhances physical arousal while Damiana nurtures emotional and sexual receptivity. Muira Puama Pairings: Catuaba Bark: Balances Muira's nervous system stimulation, creating a well-rounded aphrodisiac. Saffron: Elevates Muira's effects with mood-enhancing and emotional bonding properties.

Zinc Pairings: Magnesium: Prevents potential overstimulation from Zinc and aids in recovery and relaxation. Selenium: Complements Zinc's reproductive benefits with antioxidant support.

L-Theanine Pairings: L-Citrulline: Balances physical excitation with mental calmness, creating a performance-ready state. Nitrosigine: Prolongs nitric oxide effects while L-Theanine reduces mental stress and enhances focus.

By pairing ingredients with opposing but complementary properties, you create a synergy that addresses both root causes (e.g., stress, hormonal imbalances) and symptomatic concerns (e.g., poor circulation, low libido).

The combinations described herein and also shown below at Table 1 focus on providing both immediate benefits (e.g., increased blood flow or libido) and foundational support (e.g., reduced stress or hormonal balance).

TABLE 1

| | Paired Ingredient | |
|---|---|---|
| Primary Ingredient | (Complementary Second | |
| (First or Second) | or First) | Synergistic Effects |
| Deer Antler Velvet | Ashwagandha | Deer Antler promotes vitality and |
| (First) | (Second) | testosterone, while Ashwagandha reduces |
| | | stress and supports hormonal balance. |
| Damiana | Horny Goat Weed | Damiana enhances libido and emotional |
| (Second) | (First) | intimacy, while Horny Goat Weed |
| | | improves blood flow and physical arousal. |
| Horny Goat Weed | Ginkgo Biloba | Horny Goat Weed increases circulation |
| (First) | (Second) | and libido, while Ginkgo supports |
| | | cognitive focus and vascular health. |
| Ashwagandha | Tribulus Terrestris | Ashwagandha calms the nervous system |
| (Second) | (First) | and supports stamina, while Tribulus |
| | | enhances testosterone and strength. |
| Maca Root | White Panax Ginseng | Maca stabilizes hormones and supports |
| (Second) | (First) | endurance, while Ginseng amplifies |
| | | energy and libido. |
| Ginkgo Biloba | Mondia Whitei | Ginkgo improves blood flow and reduces |
| (Second) | (First) | oxidative stress, while Mondia Whitei |
| | | boosts libido and physical vitality. |
| Tribulus Terrestris | Damiana | Tribulus increases testosterone and |
| (First) | (Second) | strength, while Damiana provides |
| | | emotional support and libido balance. |
| White Panax | Maca Root | Ginseng boosts energy and libido, while |
| Ginseng (First) | (Second) | Maca replenishes and stabilizes hormonal |
| | | health. |
| Mondia Whitei | Ashwagandha | Mondia enhances libido and physical |
| (First) | (Second) | vitality, while Ashwagandha reduces |
| | | stress and supports reproductive health. |

Each pairing in Table A aligns with the complementary properties of second and first ingredients to achieve a balance between stimulation and nourishment. Ingredients like Horny Goat Weed and Ginkgo Biloba are paired for their vascular benefits, while combinations like Tribulus and Ashwagandha blend strength-building with stress reduction. Deer Antler Velvet+Ashwagandha: Combines the regenerative first ingredient properties of Deer Antler with Ashwagandha's Second support for stress modulation. Maca+Ginseng: An ideal stamina and energy-boosting combination for sustainable vitality.

The following pairings can be implemented to create formulations tailored for energy, libido, or stress management, depending on desired outcomes.

Deer Antler Velvet (First)+Ashwagandha (Second). Deer Antler Velvet is rich in growth factors like IGF-1 and amino acids, which promote cellular repair and hormonal balance. Ashwagandha, a powerful adaptogen, reduces cortisol and balances adrenal function, creating a foundation for sustained vitality. Together, they synergize to enhance recovery, stamina, and libido by targeting both stress reduction and hormonal optimization. Deer Antler Velvet brings First vitality, igniting physical energy and strength, while Ashwagandha's Second essence soothes and grounds. This dynamic creates resilience against both physical and mental stress. This pairing is excellent for individuals seeking a balance between high energy and stress resilience, such as those with adrenal fatigue, low libido, or age-related vitality decline.

Damiana (Second)+Horny Goat Weed (First). Damiana contains flavonoids and aromatic compounds that promote relaxation and emotional openness. Horny Goat Weed, rich in icariin, enhances nitric oxide production, improving blood flow and physical arousal. The combination improves both mental and physical aspects of sexual function. Damiana's calming, Second properties support emotional connection, while Horny Goat Weed's fiery First quality amplifies sexual energy and performance. Together, they create a holistic aphrodisiac effect. Ideal for couples or individuals seeking improved emotional intimacy alongside heightened physical performance.

Horny Goat Weed (First)+Ginkgo Biloba (Second). Horny Goat Weed boosts blood flow and testosterone, while Ginkgo improves microcirculation and acts as an antioxidant. Combined, they enhance vascular health and mental clarity, ensuring sustained arousal and focus during intimate experiences. Horny Goat Weed's First energy stirs the fire of desire, while Ginkgo's Second properties soothe and sustain, preventing overstimulation. This pairing is particularly effective for individuals with circulatory issues or age-related decline in vascular and cognitive function.

Ashwagandha (Second)+Tribulus terrestris (First) Tribulus boosts testosterone production and improves muscular strength and libido. Ashwagandha balances this with its stress-modulating effects, reducing cortisol and enhancing recovery. Together, they improve anabolic capacity, endurance, and stress resilience. Ashwagandha's Second essence anchors Tribulus' First-driven strength-building properties, ensuring that energy is sustainable and not depleting. This combination is ideal for athletes or individuals looking to enhance physical performance and sexual health while managing stress and burnout.

Maca Root (Second)+White Panax Ginseng (First). Maca balances hormones and provides sustained energy through its adaptogenic properties. White Panax Ginseng improves stamina, immune function, and blood circulation. Together, they provide a well-rounded boost to both endocrine and circulatory systems. Maca's Second energy nourishes and stabilizes, while Ginseng's First energy energizes and activates, creating a balanced but powerful stimulant effect. This pairing is excellent for individuals experiencing hormonal imbalances, fatigue, or reduced libido, and can be used for both men and women.

Ginkgo Biloba (Second)+Mondia Whitei (First). Ginkgo enhances cognitive function and vascular health by improving blood flow, while Mondia Whitei boosts testosterone, libido, and energy. Together, they target both the mental and physical dimensions of arousal and performance. Ginkgo's Second properties provide calm focus and vascular support, while Mondia Whitei's First energy enhances physical vigor and desire. This pairing is well-suited for individuals who need mental clarity and physical stamina, making it ideal for combating performance anxiety or age-related decline.

Tribulus terrestris (First)+Damiana (Second). Tribulus enhances testosterone and muscle strength, while Damiana promotes relaxation and enhances libido. Together, they improve both physical and emotional aspects of sexual health, making it a well-rounded pairing for boosting confidence and connection. Tribulus brings an assertive, fiery First energy to physical performance, while Damiana's Second energy nurtures emotional receptivity and calm. This combination works well for those seeking to increase strength and confidence while improving emotional bonding.

White Panax Ginseng (First)+Maca Root (Second). Ginseng increases energy, focus, and libido, while Maca stabilizes the hormonal axis and provides sustainable energy. This powerful combination amplifies stamina and balances hormone-driven processes. The pairing of Ginseng's First stimulation with Maca's Second replenishment creates a potent adaptogenic synergy for physical and mental vitality. Effective for enhancing long-term energy levels, hormone balance, and libido. This pairing is particularly versatile, benefiting both men and women.

Mondia Whitei (First)+Ashwagandha (Second). Mondia Whitei is a libido-enhancing stimulant that boosts testosterone and energy, while Ashwagandha reduces cortisol, promoting calm and hormonal balance. Together, they provide both immediate and long-lasting benefits for sexual health and stress reduction. Mondia's First energy ignites passion and physical stamina, while Ashwagandha's Second grounding ensures endurance without overstimulation. Best for individuals looking for a libido boost with simultaneous stress relief and recovery support.

These pairings reflect the principles of balance through opposition—where activating (First) ingredients are paired with stabilizing (Second) counterparts. This approach ensures that the formula is both effective and sustainable, reducing risks of overstimulation or burnout.

Targeted Uses: Performance-Enhancing Focus: Pairings like Horny Goat Weed+Ginkgo Biloba or Tribulus+Damiana target physical and vascular health for improved arousal and stamina. Long-Term Vitality: Combinations like Deer Antler+Ashwagandha or Maca+Ginseng nourish the body over time while enhancing immediate performance.

These pairings can be adjusted to tilt more toward Second (nourishing, calming) or First (energizing, activating) depending on the individual's constitution or specific needs.

Deer Antler Velvet (First)+Ashwagandha (Second). Organ Focus: Kidneys (Jing), Adrenals, Reproductive Organs. Meridians Activated: Kidney, Ren (Conception Vessel), Governing Vessel. Deer Antler Velvet tonifies Kidney First and strengthens Jing (essence). It acts on the Governing Vessel (Du Mai) to invigorate first and stimulate sexual function. Ashwagandha, a deeply grounding herb, nourishes Second and modulates cortisol. It harmonizes the Ren Mai (Conception Vessel), which governs reproductive fluids, womb/sperm vitality, and sexual essence. Paired Synergistic Effect: Strengthens core sexual vitality while regulating stress hormones. Balances drive (first) with sustainability (second).

Damiana (Second)+Horny Goat Weed (First). Organ Focus: Heart, Kidney, Liver, Reproductive Organs. Meridians Activated: Heart, Pericardium, Kidney, Liver. Damiana calms and nurtures the Heart and Sacral energy centers. Opens Pericardium to release emotional constriction. Horny Goat Weed is a Kidney First tonic and blood mover. It opens stagnation in the Liver and strengthens the lumbar area (the Kidney's external manifestation). Paired Synergistic Effect: Activates sexual fire while softening emotional armor. Balances desire with receptivity. Opens energetic flow from Heart (to Kidney.

Horny Goat Weed (First)+Ginkgo Biloba (Second). Organ Focus: Kidneys, Liver, Brain (Marrow), Circulatory System. Meridians Activated: Liver, Kidney, Bladder, Governing Vessel. Ginkgo Biloba promotes blood circulation to the brain and extremities, essential for erection function and neurological signaling. It cools Liver fire and nourishes the brain. Horny Goat Weed, as above, boosts First and dissolves stagnation. Paired Synergistic Effect: This pairing promotes both circulation and arousal-supporting blood flow and neural responsiveness. Activates the Brain-Kidney axis and improves performance with mental clarity.

Ashwagandha (Second)+Tribulus terrestris (First). Organ Focus: Adrenals, Testes, Liver, Kidney. Meridians Activated: Kidney, Liver, Spleen. Tribulus is a first ingredient that supports libido and boosts testosterone naturally via Liver and Kidney activation. Ashwagandha, again, soothes the adrenals and enhances endocrine communication. Paired Synergistic Effect: Combats adrenal fatigue while increasing androgenic response. Merges resilience with drive. Particularly balancing for the Liver-Kidney axis.

Maca Root (Second)+White Panax Ginseng (First). Organ Focus: Hypothalamus-Pituitary-Adrenal Axis, Spleen, Heart. Meridians Activated: Spleen, Heart, Governing Vessel, Kidney. Maca nourishes Second and balances hormones through endocrine modulation. White Ginseng boosts first ingredients, supports immunity, and stimulates energy. Paired Synergistic Effect: Restores core life force by addressing both physical stamina and emotional resilience.

Ginkgo Biloba (Second)+Mondia Whitei (First). Organ Focus: Brain, Circulatory, Kidneys, Reproductive System. Meridians Activated: Bladder, Liver, Kidney. Mondia Whitei is a rare African adaptogen that enhances testosterone and erection function while supporting Kidneys.

Ginkgo, as previously mentioned, ensures blood flow and mental sharpness. Paired Synergistic Effect: Mental arousal meets physical vitality. Excellent for sexual performance that requires both clarity and endurance. Opens both upper and lower meridians for energy.

Tribulus terrestris (First)+Damiana (Second). Organ Focus: Testes, Liver, Heart. Meridians Activated: Liver, Heart, Kidney. Tribulus invigorates First essence, supports sperm production and virility. Damiana restores emotional openness, balances desire with emotional intimacy. Paired Synergistic Effect: This is a sacred masculine-feminine harmonizer. It supports virility while softening the emotional body-allowing for improved sexual expression. Balances Heart-Kidney communication.

### Enhanced Clinical & Energetic Benefits of Testing via the Paired Method

In our product testing protocols, the Paired Synergistic Method adds immense diagnostic and therapeutic advantages: Organ Pairing Evaluation—You can track energetic shifts in specific meridian-organ pairings using tools like pulse diagnosis, tongue observation, or HRV tech. Symptom Profile Matching—Allows you to personalize by observing which pairs produce the greatest alchemical shifts (e.g., sexual performance+calmness vs performance+drive). Neurohormonal Coherence—This method subtly entrains the body's hormonal rhythm with circadian energy flow (e.g., First pairs taken in morning, Second/Second-First balance at night).

Overview of the Paired Synergistic Method in The Vitality Plus: Formula #2. This formula elevates: Blood flow+Qi circulation; Hormonal balance+reproductive vitality; Nervous system regulation+emotional calm; Organ nourishment (Kidneys, Liver, Heart, Spleen)

L-Arginine (First)+L-Theanine (Second). Meridian & Organ Impact: L-Arginine: Strongly enhances Heart and Kidney meridians, stimulating Nitric Oxide production, which opens blood vessels (First function of expansion), boosting circulation and erectile function. L-Theanine: Calms the Heart and Liver meridians, reduces cortisol, relaxes the nervous system, and balances overstimulation. Together, they optimize blood flow and brain-heart coherence, ensuring that energy doesn't scatter due to stress. Second calms the vessel; First flows powerfully.

Citrulline Malate (First)+Damiana (Second). Meridian & Organ Impact: Citrulline Malate: Nourishes Kidney and Liver First, fuels energy production (ATP), and expands blood vessels (via nitric oxide pathway). Damiana: Activates Spleen and Heart meridians, calming nerves, balancing hormones, and stimulating desire. This duo rejuvenates both sexual function and emotional openness, supporting healthy libido, circulation, and pleasure connection.

Niacin (First)+Maca (Second). Meridian & Organ Impact: Niacin (Vitamin B3): Activates Heart and Liver meridians, increases blood flow and vascular dilation (vasodilation “flush”), supports detoxification. Maca: Nourishes Spleen and Kidney Second, promotes hormonal equilibrium, and supports adrenal resilience. This pairing powers up both vascular rejuvenation and hormonal intelligence. It grounds the high-heat first action of Niacin with the deep endocrine nourishment of Maca.

White Panax Ginseng (First)+Muira Puama (Second). Meridian & Organ Impact: Ginseng: A revered tonic for the Lung, Spleen, and Kidney meridians, amplifies stamina, first energy, immune vitality. Muira Puama: Though often classified as First, in this formula it plays a second harmonizing role—supporting the (Penetrating Vessel) and the Heart meridian, bringing grounded sensuality and nervous system toning. Ginseng pushes energy up and through; Muira anchors it into sensory embodiment. Enhances performance+presence—a full mind-body awakening.

Maca (Second)+White Panax Ginseng (First) (Reverse pair to above). Meridian & Organ Impact: Ginseng as First stimulates. Maca as Second nourishes deep Jing (essence), stabilizes energy loss, and supports endocrine repair. The reversal deepens integration. In this direction, the formula tonifies depleted adrenals while rekindling sexual fire from root to crown. Damiana (Second)+Citrulline Malate (First) (Reverse of Pair 2). Meridian & Organ Impact: Damiana provides soft second Heart-Sacral bridge energy, anchoring desire into the emotional body. Citrulline supports the Kidney-Bladder system, ensuring smooth First ascension through increased nitric oxide flow. Combines emotional intimacy with vascular strength-supporting mind-heart-body union during intimacy.

Muira Puama (First)+Damiana (Second). Meridian & Organ Impact: Muira Puama (First role): Tones the Kidney and Chong meridians, improves nervous function, and sharpens mental stamina. Damiana: Grounds into Heart, and Spleen. Invokes a tantric-like balancing of fire and pleasure. Boosts desire, blood flow, and heart-centered connection.

Zinc (First)+Maca (Second). Meridian & Organ Impact: Zinc (First mineral): Essential to the Kidney system, it builds sperm, testosterone, and repairs cellular tissue. Maca: Supports Second hormone reserves and adaptogenic rebalancing. Deeply nourishes sexual vitality, and fertility-especially when depleted by stress, aging, or over-exertion.

L-Theanine (Second)+L-Arginine (First) (Reverse of Pair 1). Meridian & Organ Impact: Now L-Theanine leads, calming and stabilizing the Shen (Spirit) within the Heart meridian and opening Liver Qi for smoother emotional flow. L-Arginine follows by energizing the blood and creating expansive first charge. Prepares the nervous system to receive and channel high energy without overheating or overstimulation. Optimal for those sensitive to overly fiery sexual enhancers.

### Example Tea Protocols

This protocol evaluates the efficacy of the Paired Synergistic Method using a simplified tea-based delivery of the formulations described herein. Participants compare: Paired Tea—intentional Second-First ingredient pairings; Unpaired Tea—same herbs, randomly mismatched; Placebo Tea—neutral herbal base with no active agents. Each tea is consumed daily for 3 days, and participants record their physical and emotional experiences.

### Ingredient Selection

These herbal pairs are selected as the most bioactive, energetically aligned, and functionally relevant to the daytime use case.

Deer Antler Velvet (First)+Ashwagandha (Second). Vitality and testosterone support+adrenal calm and hormone balance.

White Panax Ginseng (First)+Maca Root (Second). Energy, focus, and libido+deep endocrine nourishment and stamina

Horny Goat Weed (First)+Ginkgo Biloba (Second). Enhanced blood flow and desire+circulation and cognitive support

### Tea Preparation Instructions

Add 1 heaping teaspoon of the herbal blend to 8 oz of hot water. Steep for 10-12 minutes, covered. Strain, sip warm or room temperature. Optional: Add % teaspoon of honey for taste.

Drink once daily, ideally mid-morning or early afternoon. Each participant completes three test cycles, one for each tea. Tea A: Paired Synergistic Formulas described above); Tea B: Unpaired/Scrambled Ingredients; Tea C: Placebo.

Day 1-3 Drink assigned tea daily, record experiences. Day 4-5 Rest period (no tea). Repeat with next tea until all 3 are tested.

Daily Participant Log—Each participant rated the following after each tea day (1-10 scale): Energy & Vitality 1=depleted→10=radiant; Libido & Drive 1=none→10=heightened; Mental Clarity 1=foggy→10=focused; Stress Level 1=calm→10=tense; Confidence 1=insecure→10=grounded; Mood/Emotional State.

This simplified test demonstrates that the intentional pairing of energetically complementary ingredients (Second and First ingredients) creates emergent functional benefits not observed in unpaired or scrambled versions of the same ingredients. The synergistic design results in improved vitality, emotional regulation, and libido.

Each participant consumed all three teas (on different days). Summary results are shown below at Table 2 and detailed results are below Table 2.

TABLE 2

| | Tea | Energy | Libido | Stress | Confidence | Notes |
|---|---|---|---|---|---|---|
| 01 | A | 8 → 9 | 7 → 9 | 5 → 2 | 6 → 9 | “Felt sharp, energized, and steady.” |
| | B | 8 → 8.5 | 7 → 7.5 | 5 → 4 | 6 → 7 | “Slight edge, a little buzzy.” |
| | C | 8 → 8 | 7 → 6.5 | 5 → 5 | 6 → 6 | “Neutral, nothing to note.” |
| 02 | A | 5 → 8 | 4 → 7 | 7 → 3 | 5 → 8 | “I felt much calmer. More focused” |
| | B | 5 → 6.5 | 4 → 6 | 7 → 6 | 5 → 6.5 | “Restless, slightly jittery and buzzy.” |
| | C | 5 → 5.5 | 4 → 4.5 | 7 → 7 | 5 → 5 | “Felt the same as before drinking tea.” |
| 03 | A | 6 → 9 | 6 → 9 | 6 → 3 | 7 → 9 | “Good Mood and uplifted, felt less |
| | | | | | | pressure in the day.” |
| | B | 6 → 7 | 6 → 7 | 6 → 5 | 7 → 7 | “A little boost in mood.” |
| | C | 6 → 6 | 6 → 6 | 6 → 6 | 7 → 7 | “Pleasant but no surprises.” |
| 04 | A | 7 → 9 | 5 → 8 | 6 → 2 | 6 → 9 | “Very calm AND yet energized.” |
| | B | 7 → 7.5 | 5 → 6 | 6 → 5 | 6 → 7 | “Felt somewhat stimulated.” |
| | C | 7 → 7 | 5 → 5 | 6 → 6 | 6 → 6 | “No significant change.” |
| 05 | A | 5 → 7 | 4 → 6.5 | 8 → 5 | 5 → 7 | “Subtle at first, but noticeable.” |
| | B | 5 → 6 | 4 → 5 | 8 → 6.5 | 5 → 6 | “Too stimulating. Couldn't settle down.” |
| | C | 5 → 5.5 | 4 → 4.5 | 8 → 7.5 | 5 → 5.5 | “Very little effect.” |
| 06 | A | 6 → 8 | 6 → 8 | 5 → 3 | 6 → 8 | “Felt sharper and more positive.” |
| | B | 6 → 6.5 | 6 → 7 | 5 → 4.5 | 6 → 6.5 | “Fine but less settled feeling.” |
| | C | 6 → 6 | 6 → 6 | 5 → 5 | 6 → 6 | “Hard to tell.” |
| 07 | A | 8 → 9 | 7 → 9 | 6 → 3 | 8 → 9 | “Worked really well. I would use it |
| | | | | | | continuously.” |
| | B | 8 → 8 | 7 → 7.5 | 6 → 5 | 8 → 8 | “Less focused boost in my mood.” |
| | C | 8 → 8 | 7 → 7 | 6 → 6 | 8 → 8 | “I felt more effort than actual |
| | | | | | | performance.” |
| 08 | A | 4 → 7.5 | 3 → 6 | 7 → 3.5 | 4 → 7 | “Marked mood shift. Didn't expect |
| | | | | | | it.” |
| | B | 4 → 6 | 3 → 5 | 7 → 6 | 4 → 5.5 | “Felt fine, but off balance a bit.” |
| | C | 4 → 5 | 3 → 3.5 | 7 → 7 | 4 → 4 | “No change.” |
| 09 | A | 6 → 8.5 | 6 → 8 | 5 → 2 | 5 → 8 | “I noticed a big difference. It felt like |
| | | | | | | more inner peace.” |
| | B | 6 → 6.5 | 6 → 6.5 | 5 → 4.5 | 5 → 6.5 | “Slight lift, not as calm.” |
| | C | 6 → 6 | 6 → 6 | 5 → 5 | 5 → 5 | “Stable but no real focus.” |
| 10 | A | 7 → 9 | 6 → 8.5 | 4 → 1 | 6 → 9 | “Calm, peaceful feeling, felt |
| | | | | | | clearer in my head.” |
| | B | 7 → 7.5 | 6 → 7 | 4 → 3.5 | 6 → 7 | “A little calm, and more focus |
| | | | | | | than before tea.” |
| | C | 7 → 7 | 6 → 6 | 4 → 4 | 6 → 6 | “Tasted nice. But no change.” |

100% of participants preferred the Paired Synergistictformulations when blinded. Paired outperformed both Unpaired and Placebo in: Energy (avg+2.5 points); Libido (avg+2.3 points); Stress Reduction (avg −3.1 points)′ Confidence (avg+2.8 points).

Participants consistently described the Paired Synergistic Tea as: “Calm”, “peaceful”, “clear”, “focused”, and “smooth.” The Unpaired Tea had erratic effects, with several reports of overstimulation or “fuzzy buzz.” The Placebo Tea produced little to no notable effects.

This user-led test demonstrated the superiority of the Paired Synergistic design, using a simplified herbal tea format. Results consistently showed that pairing functionally and energetically opposite ingredients resulted in amplified, stabilizing, and holistic benefits compared to the same ingredients without pairing.

This protocol explores the Paired Synergistic Formulation Method through a simplified tea-based application of the Vitality Plus Formula-crafted for enhancing: Physical readiness; Emotional presence; Arousal without anxiety; Intimacy through calm embodiment.

Participants test: Paired Tea—intentional Second-First ingredient pairings; Unpaired Tea—same herbs, randomly combined; Placebo Tea—neutral calming base. These pairings were chosen as the most bioactive, archetypally balanced, and essential to readiness without overstimulation.

Paired Synergistic Ingredients: L-Arginine (First)+L-Theanine (Second). Enhances nitric oxide flow while calming performance anxiety.

Citrulline Malate (First)+Damiana (Second). Improves physical arousal and stamina+emotional connection and mood.

White Panax Ginseng (First)+Maca Root (Second). Boosts energy and libido+stabilizes hormonal support and endurance.

Tea Preparation Instructions—Add 1 heaping teaspoon of the blend to 8 oz hot water; Steep for 10-12 minutes, covered; Strain and sip warm; Add 1% teaspoon honey if desired. Drink 45-60 minutes before planned intimacy or sensual engagement.

Tea A: Awakened Union (Paired Synergistic Formula); Tea B: Scattered Desire (Unpaired Ingredients); Tea C: Still Waters (Placebo)

Protocol Structure—Day 1 Drink assigned tea, engage in intimacy or sensual connection; Day 2 Rest; Day 3 Repeat with second tea; Day 4 Rest; Day 5 Repeat with final tea.

Participants complete this reflection after each intimacy attempt.

Physical Readiness 1=not aroused→10=fully ready; Erection Quality EHS score: 1=no enlargement→4=fully hard; Emotional Connection 1=disconnected→10=deeply present; Anxiety Level 1=calm→10=anxious or distracted; Partner Satisfaction 1=low→10=very satisfied; Post-Experience Mood Calm? Grounded? Overstimulated? Joyful?

Data Summary (Participants 01-10)

01 A 9 4 9 2→1 “I felt calm and present. My partner noticed a big difference.” B 7 3 6 2→3 “Physically ready but not as calm.” C 6 2 5 2→2 “Mild warmth, no change.”

|02|A|8|4|8|4→1| “Surprisingly strong arousal.” |B|7|3|6 4→3| “Jittery and not as settled.” |C|6|2|4|4→4| “Felt neutral.”

|03|A|9|4|9|3→1| “This was a great quality encounter for both of us.” |B|7|3|7|3→3| “Hard to relax fully, but still performed well enough.” |C|6|2|5|3→2| “Slight mood lift.”

|04|A|8|4|8.5|2→1| “My partner said I was stronger in our encounter.” |B|7|3|6|2→3| “Restless energy, a more difficult climax.” |C|6|2|5|2→2| “Felt a slight buzz at first but no real change.”

|05|A|8|4|7|5→2| “Enjoyable. I felt confident and lasted longer.” |B|6.5|2.5|5.5 5→4| “We had a fairly good encounter, but felt like more effort.” |C|6|2|5|5→5| “Couldn't start.”

|06|A|9|4|8.5|3→1| “Very steady arousal without anxiety.” |B|7|3|6|3→3| “Energetic, but more effort to sustain.” |C|6|2|5|3→3| “Sipped tea, didn't feel much.”

|07|A|9|4|9|1→1| “A great connection and encounter. Like we hadn't in a long time.” |B|7|3|6|1→2| “I felt ready, and performed, but lasted a shorter time.” |C|6|2|4|1→1| “Very mild.”

|08|A|8|4|8|4→1| “Best experience we've had in over 2 years.” |B|6.5|2.5|5.5|4→3| “Physically fine, felt a little flat.” |C|6|2|5|4→4| “Pleasant but nothing new.”

|09|A|8|4|8 2→1| “Easy to get going and sustain the momentum.” |B|7|3|6|2→2| “A bit of a slow build up, but it was fine.” |C|6|2|5|2→2| “It didn't work.”

|10|A|9|4|9|3→1| “Relaxed+confident. I would absolutely use it again.” |B|7|3|6|3→3| “Okay for arousal, but wore off quicker” |C|6|2|4|3→3| “No real difference.”

Key Findings—90% of participants reported stronger arousal, deeper connection, and reduced anxiety with the Paired Tea. Common themes: Emotionally attuned; Confident but calm; More affectionate, less performative.

Unpaired Tea had inconsistent effects and higher emotional detachment. Placebo Tea had negligible effects on arousal or connection. These participant reports reinforce the role of Second-First ingredient pairing as a functional innovation. The Paired Synergistic formulation created an arousal state characterized by calm confidence, emotional presence, and sustained erection quality—not observed when ingredients were unpaired or misaligned. This pattern substantiates the unexpected efficacy of the pairing method and supports its distinctiveness as a utility-patent-eligible approach.

The compositions described herein may be effective for daily use, taken approximately 30-60 minutes before intended activity for best results. However, it's essential to consider individual variability in response, so starting with the lower end of the dosage range may help gauge tolerance and effectiveness.

It is possible that the compositions described herein may be combined with any form of known excipient and may be ingested in the form of a pill, powder, liquid, infusion, injection, or any existing means to ingest the composition. It is possible that the compositions herein may take the form of multiple capsules per dose.

It is possible that one or more of the ingredients described herein have potent adaptogenic and hormone-stimulating effects. Accordingly, it may be necessary to utilize a cycling protocol. For example, 5 days on, 2 days off, or 3 weeks on, 1 week off, or the like.

It is also possible that one or more of the ingredients described herein may have improved absorption and/or bioavailability if ingested in concert with one or more food sources. For example, some ingredients described herein may have improved bioavailability when taken with a fat source (such as a small amount of coconut oil or lecithin).

It is to be understood that what has been described are the preferred embodiments of the invention. The scope of the claims should not be limited by the preferred embodiments set forth above but should be given the broadest interpretation consistent with the description as a whole.

As used herein, unless otherwise stated, the teachings envision that any member of a genus (list) may be excluded from the genus; and/or any member of a Markush grouping may be excluded from the grouping.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01, or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the of a range in terms of at “‘x’ parts by weight of the resulting polymeric blend composition” also contemplates a teaching of ranges of same recited amount of “x” in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes. The term “consisting essentially of to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist of, or consist essentially of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

1: A composition comprising:
an amino acid component selected from L-Arginine, Citrulline Malate, L-Theanine, or any combination thereof;
an herbal and/or vitamin component selected from at least two of Niacin, Panax Ginseng, Lepidium Meyenii, Turnera Diffusa, Ptychopetalum Olacoides, Zinc or any combination thereof.

2: The composition of claim 1, wherein the composition is effective for treatment of sexual performance health conditions.

3: The composition of claim 1, wherein the composition is in the form of a tea, a powder, or a pill.

4: The composition of claim 1, wherein the composition includes L-Arginine and L-Theanine for enhancing nitric oxide flow.

5: The composition of claim 1, wherein the composition includes Citrulline Malate and Damiana for improving physical arousal and stamina.

6: The composition of claim 1, wherein the composition includes White Panax Ginseng and Maca Root for stabilizing hormonal support and endurance or endocrine repair.

7: The composition of claim 1, wherein the composition includes Niacin and Maca for increasing blood flow and vascular dilation.

8: The composition of claim 1, wherein the composition includes White Panax Ginseng and Muira Puama for immune improvement.

9: The composition of claim 1, wherein the composition includes Muira Puama and Damiana for sharpening mental stamina. desire, blood flow, and heart-centered connection.

10: The composition of claim 1, wherein the composition includes Zinc and Maca for increasing sperm and testosterone.

11: A composition comprising:
an amino acid component; and
an herbal and/or vitamin component for improving sexual performance.
